(19) United States
(12) Plant Patent Application Publication
(10) Pub. No.: US 2013/0326772 P1
(43) Pub. Date: Dec. 5, 2013

(54) Cherry tree named 'SWEET ARYANA'

(50) ;

(71) Applicant: Alma Mater Studiorum-Universita degli Studi di Bologna, Bologna (IT)

(72) Inventors: Stefano LUGLI, Modena (IT); Riccardo CORREALE, Bologna (IT); Michelangelo GRANDI, Castenaso (BO) (IT)

(21) Appl. No.: 13/986,702

(22) Filed: May 28, 2013

(30) Foreign Application Priority Data
May 25, 2012 (QZ) 2012/1157

(51) Int. Cl. A01H 5/00

(52) U.S. Cl. PLT/181

(57) ABSTRACT

Variety Denomination PA1UNIBO, named ‘Sweet Aryana’, is a novel cherry tree derived from a seedling of unknown cross which was planted at Vignola, Modena Province, Italy. The genus and species (cultivar) of PA1UNIBO is Prunus avium. It was initially propagated by grafting to root stocks of varying vigor and, after testing in districts and plantings of differing types, proved to have the qualities proper to a promising new cultivar for the market.

### CROSS-REFERENCE TO RELATED APPLICATIONS

This application claims priority of the pending EU Plant Patent for ‘Sweet Aryana’ was filed on 25 May 2012 as Application No. 2012/1157 in the name of Applicant (holder) Alma Mater Studiorum—Università degli Studi di Bologna, the contents of which are expressly incorporated herein by reference. EU Trademark for ‘Sweet Aryana’ was released on 2 Jan. 2013 as Registration No. 011058559 of Applicant (holder) Alma Mater Studiorum— Università degli Studi di Bologna.

### LATIN NAME OF THE GENUS/SPECIES OF THE PLANT CLAIMED (CULTIVAR)

Prunus avium.

### VARIETY DENOMINATION

PA1UNIBO

### BACKGROUND OF THE INVENTION

Prunus avium PA1UNIBO ‘Sweet Aryana’ originally derived from seedling of unknown cross and called Selection DCA BO A1A1, was planted at Vignola, Modena Province, Italy, in February 2002, and was first cropped in 2004. It was initially propagated by grafting to root stocks of varying vigor, tested in different growing districts and planting densities, and proved to have the properties proper to a promising new cultivar for the market. It picks 3-5 days after ‘Burlat’ (15-13 days before ‘Bing’).

### BRIEF SUMMARY OF THE INVENTION

The parentage of the variety is unknown. The name of a comparison variety is ‘Burlat’ (e.g., ‘Bing’ ripens 18 days after ‘Burlat’). Compared to ‘Burlat’, its fruit is larger, of firmer flesh, with more uniform ripening and higher sugar content. PA1UNIBO was selected for its high qualities, including tree growth and yield performance, and fruit appearance and excellent taste-flavor properties. It also ripens earlier than its comparison varieties, has large-sized fruit, and excellent flesh firmness. Its other most distinctive traits include mid-season, uniform ripening and large, firm-fleshed fruit. The date of discovery/cross was in the year 2000. The location of the discovery was Vignola, Modena Province, Italy. The date of the first asexual propagation was in 2008 in Vignola, Modena Province, Italy. PA1UNIBO was first propagated on ‘Colt’ and ‘Gisela 6’ root stocks of differing vigor in trial fields of Bologna University, to test growth and yield performance under differing training systems and planting densities.

### BRIEF DESCRIPTION OF THE DRAWINGS

The accompanying photographs show typical specimens of the new variety as depicted in color as nearly true as is reasonably possible in color illustrations of this character.

FIG. 1 illustrates the open, semi-spreading tree architecture with fully-open blossoms of PA1UNIBO.

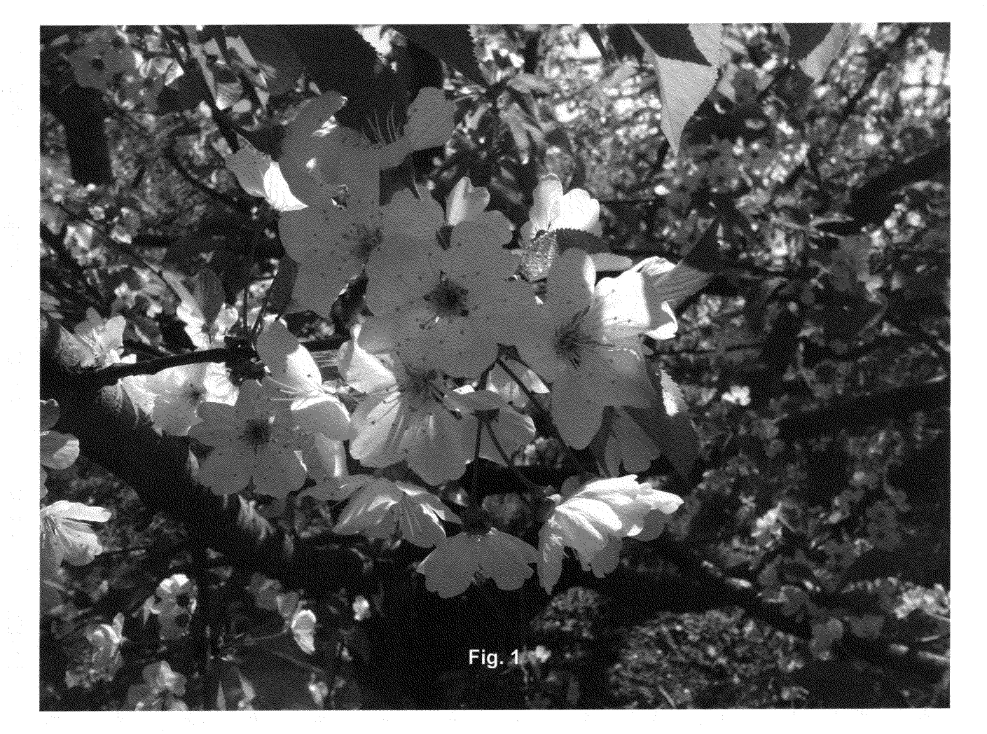

FIG. 2 is the flower, full front, in profile and with separated petals, of PA1UNIBO.

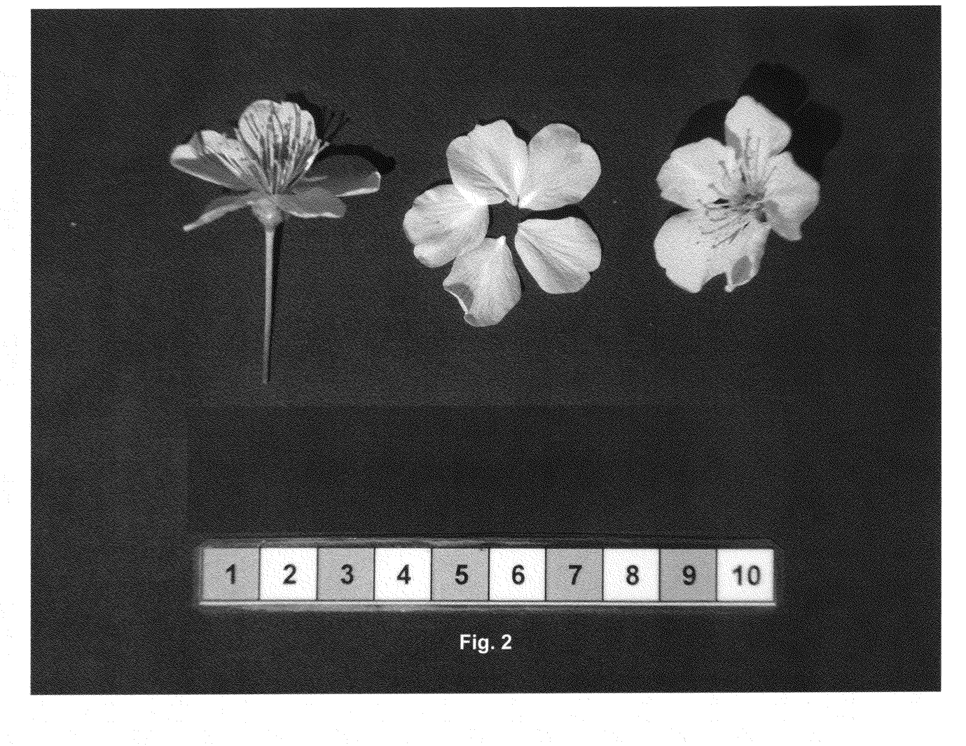

FIG. 3 shows the leaf profile of PA1UNIBO. Two leaves have been detached and one has been placed upper leaf surface side down to show the lower leaf surface details.

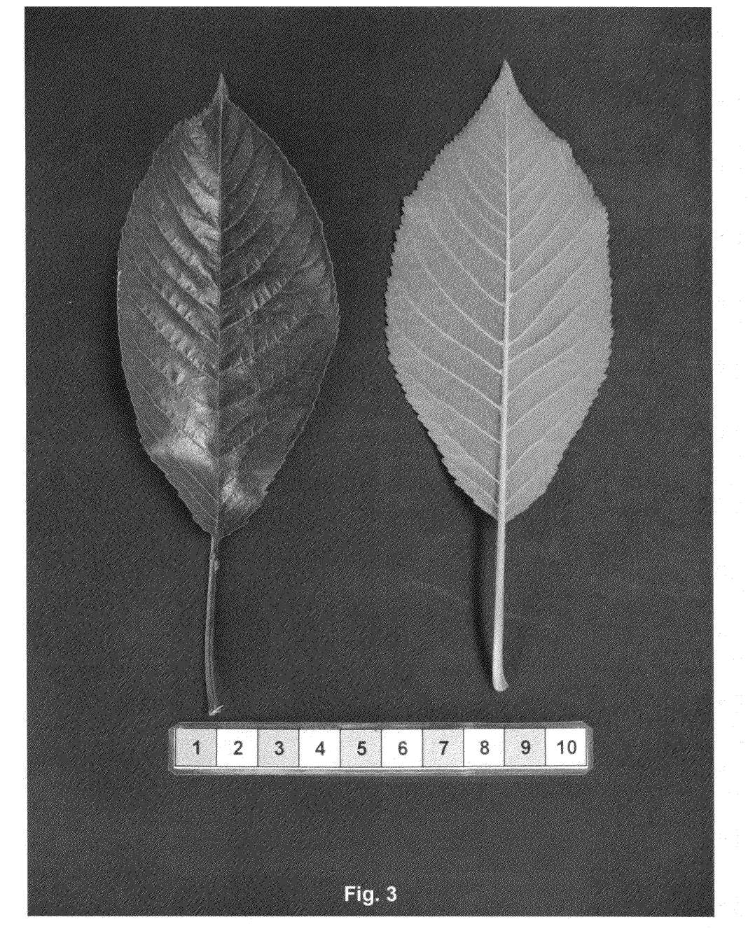

FIG. 4 is a close-up of the fruit cropping of PA1UNIBO.

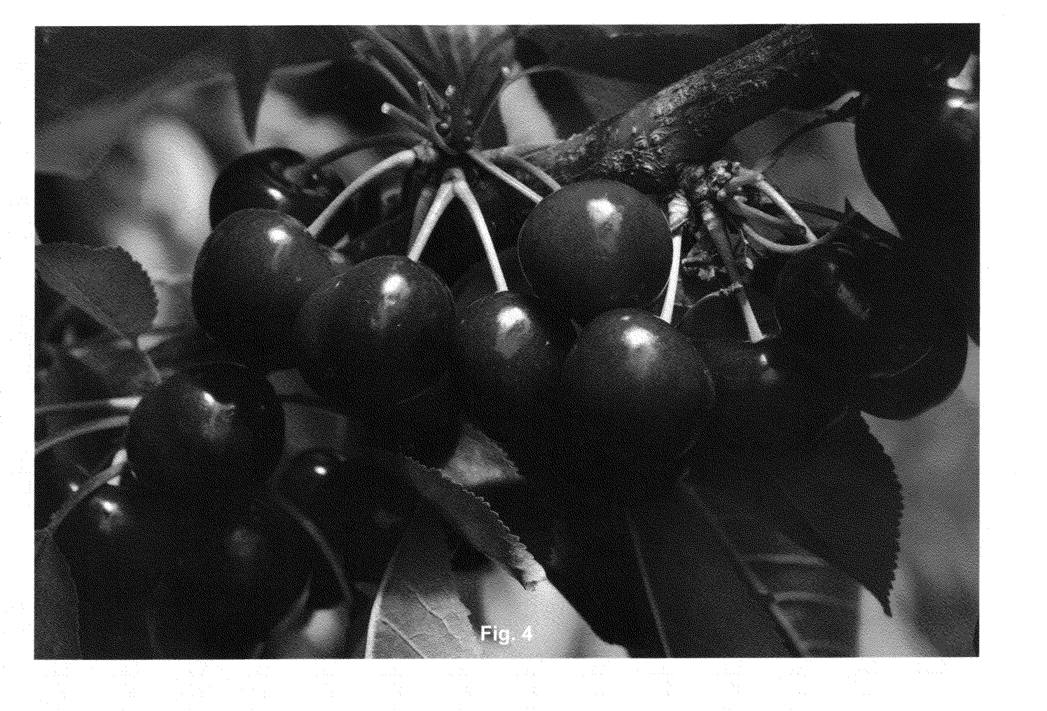

FIG. 5 shows the tree habit, with width-spread and height, of PA1UNIBO.

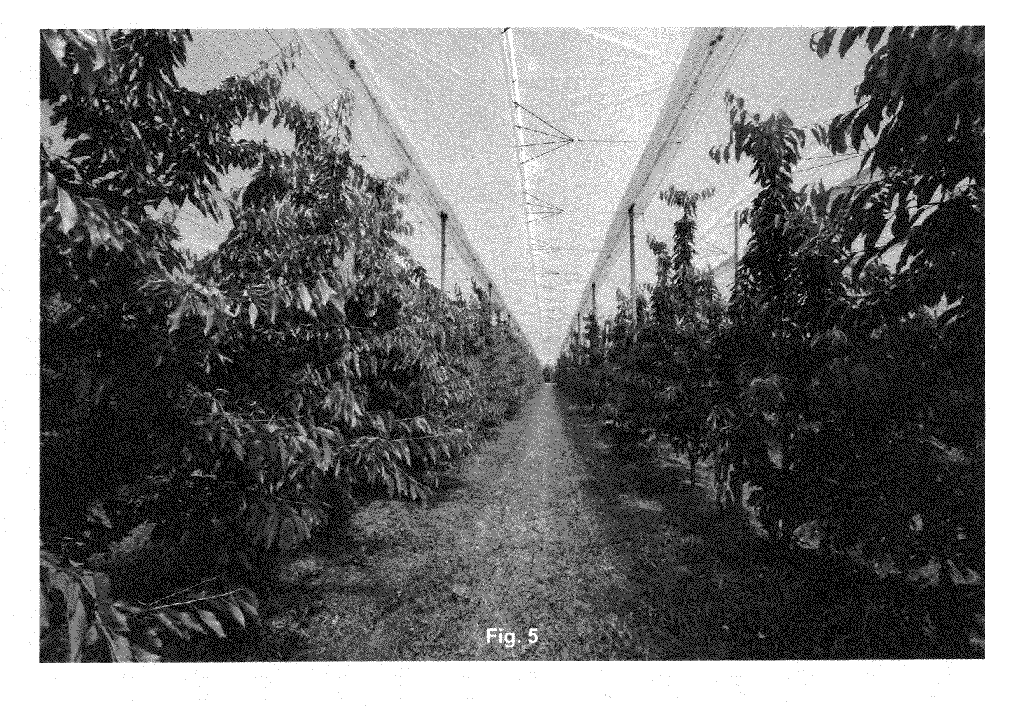

### DETAILED BOTANICAL DESCRIPTION OF THE PLANT CLAIMED

General:
   Age of described tree(s).—5 years, multi-leader spindle.
   Location of described trees(s).—Vignola, Modena Province, Italy.
   Name of color chart used.—Royal Horticultural Society Colour Chart.
   Name of root stock.—‘Colt’.
Tree:
   Vigor.—High.
   Height.—4.5 meters.
   Spread.—3 meters.
   Growth habit.—Open.
   Canopy density.—Medium.
   Trunk diameter at specified height.—16-20 cm, as measured at about 5 cm above graft joint.
   Trunk color.—Reddish-brown (200C) flecked with gray (201A).
   Bark texture.—Medium rough.
   Tendency toward alternate bearing.—No.
   Winter hardiness.—Unknown; no winter cold damage found in this Po Valley district.
   Chilling requirement.—Unknown.
Branch:
   Length.—300-400 cm.
   Diameter.—5-6 cm.
   Crotch angle.—45°-60° depending on training system.
   Bark color.—Brown (200B) flecked with red (183A).
   Bark texture.—Medium rough.
Current year shoot:
   Length.—40-65 cm.
   Color.—Reddish-brown (200C/200D) with fairly widespread gray overtone (201A).
Winter hardiness:
   USDA hardiness zone.—Unknown.
   Chilling requirement.—Unknown.
Flower buds:
   Number per spur.—4-6, but usually 5.
   Shape.—Sub-globose.
   Length.—About 1 cm.
   Diameter.—Under 1 cm.
   Color.—Reddish perules (187C).
Flowers:
   Number per cluster.—Minimum 7, maximum 9 per spur; 2-3 per bud. Bloom is abundant.
   Relative position of petals.—Petals overlap slightly.
   Diameter.—32-37 mm.
   Depth.—13-15 mm, measured as distance from receptacle to maximum petal aperture.
   Is pollen produced?.—Yes.
   Coloration of pollen.—Pale yellow (12A).
Sepal:
   Length.—5-7 mm.
   Shape.—Heart-shaped.
   Width.—About 5 mm.
   Apex.—Pointed.
   Base.—Flat.
   Margin descriptors.—Regular margin.
   Color — upper surface.—Pale green (138C).
   Color — lower surface.—Pale green (138C).
Petal:
   Number per flower.—5.
   Shape.—Circular.
   Length.—14-16 mm.
   Width.—15-17 mm.
   Apex.—Slightly sunken.
   Base.—Slightly pointed.
   Margin.—Slightly undulated.
   Color — upper surface.—White (155B).
   Color — lower surface.—White (155B).
Bloom:
   Date of bud burst.—7-8 Apr. 2013.
   Date of first bloom.—13 Apr. 2013.
   Date of full bloom.—16 Apr. 2013.
Pedicel:
   Length.—33-36 mm.
   Diameter.—About 1.2 mm.
   Color.—Pale green (143C).
Pistil:
   Quantity.—1.
   Size.—19-23 mm.
   Color.—Pale green (143D).
Anthers:
   Quantity.—22-25, with about 70% of 10-13 mm in length, positioned slightly below the top of the stigma, and the remaining 30% varying from 5-8 mm. White filament (155D).
   Size.—About 1 mm diameter.
   Color.—Orange-yellow (15A).
Stigma:
   Quantity.—1.
   Size.—1.1-1.3 mm diameter.
   Color.—Pale green (142B).
Styles:
   Quantity.—1.
   Size.—14-16 mm length.
   Color.—Pale green (142B) at basal and distal parts, white-green (142D) in center turning to rosé (36C) at end of bloom.
Ovary/ies:
   Quantity.—1.
   Size.—5-7 mm.
   Color.—Pale green (143D).
Leaves:
   Length.—11.8-12.4 cm.
   Width.—6.1-6.8 cm.
   Shape.—Lanceolate-elliptical.
   Apex shape.—Pointed.
   Base shape.—Pointed.
   Margin.—Fine toothed.
   Color — upper surface.—Dark green (137A).
   Color — lower surface.—Pale green (138A/138B) veined green-yellow (152A/152B) in center.
Petiole:
   Length.—4.1-4.5 cm.
   Diameter.—About 2 mm.
   Color.—Green-yellow lower (152A/152B) and reddish upper (183A).
Fruit:
   Shape.—Depressed heart-shaped.
   Diameter.—Mostly 28 mm.
   Height.—21-24 mm.
   Weight.—11-12 grams on average.
   Number per cluster.—5-10 per spur.
   Skin color (include ground color and over color).—Shiny dark red (187B) tending to blackish-dark red (187A) at advanced maturity.
   Lenticels present? Density?.—Yes, medium density.
   Skin thickness.—Medium-thin.
   Skin texture.—Medium.
   Anthocyanin coloration present near skin?.—No.
   Tenacity of flesh to stone.—Low.
   Flesh color.—Red (53A).
   Flesh texture.—Fine.
   Juiciness.—High.
   Juice analysis (brix, acidity, tss, etc.).—17.6 ° Brix and 8.6 g/1 malic acid (2012 data).
   Stone size.—Medium-small.
   Stone shape.—Elliptical.
   Stone color.—Pale brown (165D).
Fruit stem:
   Length.—35-38 mm.
   Diameter.—Just over 1 mm.
   Color.—Green (143A).
Harvest:
   Maturity date range.—5 days after ‘Burlat’ (13 days before ‘Bing’).
   Harvest date range (if different).—Same as above. Because of the outstanding and unique horticultural characters of the claimed plant, especially its long harvest period and large size, this cherry can be planted to extend the cultivar's season. Although this new variety of cherry possesses the above-described characteristics, it is to be understood that variations and fluctuations may occur in the magnitude and qualities due to changes in growing and climate conditions, irrigation, fertilization, pruning and pest control.

What is claimed is:

1. A new and distinct cherry tree as herein described and illustrated.
